(19) United States
(12) Patent Application Publication
(10) Pub. No.: US 2015/0182431 A1
(43) Pub. Date: Jul. 2, 2015

(54) Compositions and Methods for Treatment of Hair with Reduced Hair Damage

(71) Applicant: SYTHEON LTD, Boonton, NJ (US)

(72) Inventor: Ratan K Chaudhuri, Lincoln Park, NJ (US)

(73) Assignee: SYTHEON LTD, Boonton, NJ (US)

(21) Appl. No.: 14/145,928

(22) Filed: Dec. 31, 2013

(51) Int. Cl. A61K 8/35; A61Q 5/02; A61Q 5/04; A61Q 5/10

(52) U.S. Cl. CPC A61K 8/35; A61Q 5/10; A61Q 5/02; A61Q 5/04

(57) ABSTRACT

The present invention relates to hair care compositions comprising select benzylidene 2,4-pentanedione compounds and methods for reducing hair damage, especially oxidative hair damage, arising from various hair treatments. In particular, the compositions according to the present teachings contribute to a reduction or lessening in oxidative damage sustained by hair fibers during bleaching, repeated shampooing, dyeing, perming, straightening and other oxidative treatments. The compositions according to the present invention also provide excellent color evenness, sustained color protection, and reduce scalp itching.

This application claims the benefit of U.S. patent application Ser. No. 13/310,924 filed Dec. 12, 2011, now U.S. Pat. No. 8,617,528 which claims the benefit of U.S. Provisional Patent Application No. 61/419,961, filed on Dec. 6, 2010, now lapsed, both of which are hereby incorporated herein by reference in their entirety.

### BACKGROUND

Minerals such as calcium, magnesium and copper hold on to one's hair every time one washes it with tap water. Calcium and magnesium are relatively benign; however, copper is not. In the highly alkaline environment of ammonia-based hair dyes (normally a pH of 10 or 11) copper can react with hydrogen peroxide to form highly reactive hydroxyl radicals.

There are two key reactive species in permanent hair color: the perhydroxyl anion (HOO⁻) and the hydroxyl radical (HO⁻). While HOO⁻ has some damaging side effects, it is the species that is responsible for the desired changes to hair color. The HO⁻ radical, on the other hand, is an undesired by-product of a reaction between H₂O₂ and metal ions, especially copper. It does not contribute to the development of color but does contribute to hair damage. It is believed that the elimination of this unnecessary free radical can significantly reduce hair damage associated with hair coloring without compromising hair colorant performance. One approach is to reduce the formation of free radicals by eliminating the exposure to metal ions.

Repeatedly dyeing hair can strain and gradually destroy the hair cuticle—leaving hair harder to control, even as determined brushers become unwitting accomplices in destroying more of the unstable shingle-like cells through vigorous brushing. With less of the cuticle to stop them, free radicals are free to attack the protein fibers within the cortex by forcing apart their structural supports and fraying their ends: hence, the manifestation of split ends.

In this regard, it's a bit of a vicious cycle. With every dye treatment, the hydroxyl radicals attack the hair's waterproofing layer and make it increasingly porous, allowing more copper to get in during the next shampoo. People who repeatedly color their hair, in fact, end up accumulating significant copper levels and compounding the damage. Even if one doesn't color the hair, a small amount of copper from the tap water still accumulates which can react with other constituents of hair treatment products leading to increased damage.

So how can regular dyers stop the buildup of copper? One strategy is to take out or effectively neutralize the mineral by binding it to something else. However, with far more calcium and other minerals around, that binding compound would have to be very selective. Generally speaking, there is from 10 to 100 times more calcium present in the water than copper and, as a result, in hair as well. If one selects a binding compound that reacts with both, it would essentially react with all the calcium before it saw any of the copper.

It is well documented that EDTA, although widely used in lotion, cream, shampoo, bleaching and dyeing compositions and a known chelating agent, displays very little benefits in addressing hair damage arising from copper. Indeed, it has been found to enhance oxidative damage by the following reaction:

Cu²⁺-EDTA+H₂O₂→Cu⁺-EDTA+O₂⁻+2H⁺

### (Chaudhuri RK & Puccetti G, Transition Metal-Induced Oxidation: Implications for Skin Care Products, C&T, 117(9), p. 43-56, 2002).

Given the wide use of hair colorants and other hair treatments which adversely affect hair, there have been numerous attempts to protect the hair from damage instead of merely concealing it.

U.S. Pat. No. 4,138,478 discloses compositions for changing the color of hair by the action (direct or indirect) of compounds therein which release nascent oxygen which compositions do less harm to the hair, as compared to other then state of the art coloring compositions, when they have a content of select water-soluble 3-amino-1-hydroxypropane-1,1-diphosphonic compounds. The select aminohydroxypropane-diphosphonoic compounds can be in free acid or water-soluble salt or partial ester form. Hair which is pre-treated with one of the mentioned diphosphonic compounds is at least partially protected against the action of nascent oxygen. According to this patent, “the diphosphonic compound is substantively adsorbed by the hair and acts to hinder degradation of the hair by nascent oxygen which is either present therewith or which is substantially added.” Other protective compounds such as hydroxyethane-1,1 diphosphonic acid (HEDP) and ethylenediaminetetramethylene phosphoric acid (EDTMP) are disclosed at low levels in U.S. Pat. No. 3,202,579 and U.S. Pat. No. 3,542,918.

U.S. Pat. No. 5,100,436 discloses a method of dyeing hair wherein the hair is pretreated with an aqueous solution containing select metal-chelate complexes and then dyed with an oxidative dye mixture. The use of catalytic amounts of select transition metal ions complexed with dipyridyl or o-phenanthroline chelating agents (0.001 to 0.1% by weight of the solution) allows a reduction in the time of exposure, thus reducing the damage caused by the oxidizing agent.

U.S. Pat. No. 5,635,167 discloses a process for the removal of exogenous minerals which have become attached to human hair or keratin fiber, the process including the steps of (i) contacting at least one chelating agent to the human hair or keratin fiber, wherein the chelating agent is selected from the group consisting of amino acid-type, polyphosphate-type and phosphonate-type agents, (ii) maintaining contact of the chelating agent with the human hair or keratin fiber for a period of time sufficient to permit the chelating agent to complex with at least a portion of the exogenous minerals, and (iii) rinsing the chelating agent from the hair or keratin fiber thereby removing the attached minerals. The process is enhanced when the pH is adjusted to a range of between 4 to 9, preferably 5 to 8. The chelating agent is added at a concentration of 4% by weight to 25% by weight, preferably 5 to 20% by weight. In a preferred case, the chelating agent is selected from the group consisting of a salt of ethylene-diaminetetraacetic acid, a salt of hydroxyethylethylenediaminetnacetic acid, a salt of diethylenetriaminepentaacetic acid, a salt of nitrilotriacetic acid and a salt of tripolyphosphate, preferably the sodium salt. The chelating agents used are preferably blends of chelating agents in an effort to achieve a combined or, more preferably, a synergistic effect.

U.S. Pat. No. 6,013,250 discloses hair treatment compositions which provide an excellent finishing effect and superior protection against environmental, chemical, and grooming-associated damage. These treatment compositions comprise hydrolyzed protein having an abundance of anionic amino acids and, in particular, sulfur-containing amino acids, as well as divalent cationic compounds. It is said that the anionic components of the hydrolyzed protein effectively bind to the hair by means of cationic bridges. While bound to the hair, the sulfur containing amino acids in the hydrolyzed protein are said to serve as “decoys” for the effects of a variety of damaging agents.

U.S. Pat. No. 7,179,302 discloses that ethylenediamine-NN-disuccinic acid (EDDS) and derivatives and salts thereof have little attraction for calcium but chelate onto copper, thereby leaving it unavailable for radical making. Preferred salts include the alkali metal, alkaline earth metal, ammonium and substituted ammonium salts (e.g. monoethanolammonium, diethanolammonium, triethanolammonium). It is said that these compounds reduce the oxidative damage sustained by keratinous fibers, such as hair, during bleaching, dyeing, perming or other oxidative treatments.

U.S. Pat. No. 7,186,275 discloses hair care compositions comprising specific chelants and the use thereof for reducing oxidative hair damage during oxidative treatments of hair such as bleaching, oxidative dyeing or perming. Suitable chelants include diamine-N,N′-dipolyacids and monoamine monoamide-N,N′-dipolyacids.

WO97/24106 discloses hair coloring compositions comprising a water soluble peroxygen-bleach, a bleaching aid selected from organic peroxyacid bleach precursors and preformed organic peroxyacids, and one or more hair coloring agents. Various chelants are disclosed as optional ingredients and exemplified in hair care compositions at 0.1% by weight of the composition. The organic peroxy acid bleach precursors are defined as organic compounds that react with hydrogen peroxide in a perhydrolysis reaction to produce a peroxyacid. These bleaching aids are claimed to provide benefits including reduced hair damage at lower pH. However, at a pH higher than 8, the present Applicant has found that these bleaching aids are more damaging to hair than usual water-soluble oxidizing agents such as hydrogen peroxide. Without being bound by theory, the present Applicant believes that the conjugate base of the organic peroxyacid formed at a pH above 8 is more likely to oxidize the disulphur bonds of the keratin than other oxidizing agents such as hydrogen peroxide. Additionally, hair coloration, especially with oxidative dyes, is much poorer at pH 8 than pH 10. Finally peroxyacid precursors are difficult to solubilize, especially in oil-in-water emulsion.

Recent advances to enhance color retention and/or reduce color fading of artificially colored hair have included the use of color-protecting agents. The color-protecting agents may include mild surfactants, cationic conditioning agents, aminofunctional silicones, ultraviolet absorbers, starches and sugar surfactants and are typically incorporated into hair care compositions through emulsification, dissolution and other methods know to make them compatible with such hair care product compositions. For example, U.S. Pat. No. 5,922,310 describes a composition for preventing the fading of artificial hair dye and/or slowing down the oxidation of hair comprising a cationic antioxidant phenol.

U.S. Pat. Nos. 6,129,909 and 6,180,091 describe a hair-treating composition comprising a diester or polyester of a naphthalene dicarboxylic acid for imparting gloss and/or hair color stabilization. It is stated that the naphthalene dicarboxylic acid can be mixed in an aqueous phase of the hair-treating composition to impart improved photo-stabilizing properties.

U.S. Pat. No. 5,045,307 describes a method of treating dyed hair to reduce color loss caused by exposure to the ultraviolet rays of the sun by applying an effective amount of a hair treating formulation comprising an effective amount of a water insoluble benzophenone compound in combination with a compatible hair substantive carrier composition to the hair.

U.S. Pat. No. 6,143,286 describes hair conditioning compositions comprising a cationic conditioning agent, a fatty alcohol, a nonionic surfactant and a particular siloxane polymer having both difunctional units and trifunctional units, in a ratio of about 10 to 80 difunctional units for every trifunctional unit.

U.S. Patent Publication 2005/0188481 describes a composition comprising at least one amylose-containing starch, and its use in extending or improving the color durability and stability of artificially colored hair.

Additionally, a number of hair dye manufacturers have begun to incorporate color-protecting agents in “color sealing” conditioners to be used after the first shampooing of newly artificially colored hair. However, the use of such conditioners cannot provide enhanced color retention and/or reduced color fading during the first shampooing. Rather, during the first shampooing, the newly artificially colored hair is not protected from water or shampoo and a noticeable amount of the chemical dye molecules are leached from the hair at that time. This causes the newly artificially colored hair to suffer a substantial amount of color fading before a color-protecting agent is applied.

Despite the advances for color-protecting agents, there remains a need in the art for enhanced color retention and/or reduced color fading of artificially colored hair. It is therefore an object of the invention to provide compositions and methods for imparting enhanced color retention and reduced color fading to artificially colored hair.

In addition to the issues with color fading, there is also the earlier mentioned continued concern with damage to hair caused by the strongly aggressive chemicals contained in most bleaching, copper chelating, dyeing or perming compositions, particularly with repeated usage. Despite the claims, especially in the trade literature, to the contrary, our studies have shown that EDDS is not an effective chelating agent for copper and, thus, is not effective in combating hair damage associated therewith.

Thus, not only is there a need for compositions which provide better protection of and longevity to hair coloration, there is still an urgent an strong need for effective treatments for addressing hair damage arising from hair coloration and other hair treatment applications. Specifically, despite the many developments noted above and available in the marketplace, there's still plenty of room for improvement in developing hair- and scalp-friendly products.

In following, it an object of the present invention to provide new hair care compositions capable of improved protection of human hair from oxidative damage, particularly the structurally important keratin bonds, such as the disulphide bonds, human hair from oxidative breakage.

It is another object of the present invention to provide hair care products that improve hair color evenness and/or longevity, most preferably with less scalp irritation.

It is another object of the present invention to provide methods of treating hair for reducing oxidative hair damage and other hair damage, especially that arising from hair coloration, bleaching, perming, shampooing, etc.

It is another object of this invention to provide bleaching, dyeing or perming and shampooing compositions with a better efficiency in terms of light shade, color evenness, color fading and hair feel.

It is another object of this invention to provide bleaching or dyeing compositions capable of protecting hair fibers while at the same time delivering a good lightening/coloring effect.

### SUMMARY

According to the present teachings, it has now been found that various benzylidene 2,4-pentanedione compounds are very effective in chelating iron and copper, more so than EDDS and EDTA. In following, it has now been found that these compounds are very effective in protecting hair fiber from damage during oxidative treatment, either as a pretreatment, as a post treatment, or as a component of the oxidative treatment composition itself. In this later case, the present teachings provide many, if not most, of the desired attributes of the utopian, or nearly utopian, bleaching, dyeing or perming and shampooing composition and also provide said compositions with better efficiency and efficacy in terms of light shade, color evenness, color fading and hair feel.

Specifically, according to the present invention there are provided hair treatment compositions and methods which incorporate one or more benzylidene pentanedione compounds according to the Formula I as follows:

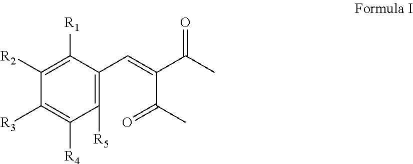

wherein R₁, R₂, R₃, R₄ and R₅ which may be the same or different, are independently H, alkyl or alkoxy, preferably H, alkyl or alkoxy, wherein the alkyl or alkoxy groups are linear or branched and have from 1 to 8 carbon atoms, preferably from 1 to 6 carbon atoms, more preferably from 1 to 4 carbon atoms, with, preferably, at least one of R₁, R₂, R₃, R₄ and R₅ being other than H. Most preferably, R₁, R₂, R₃, R₄ and R₅ are H, methyl or methoxy, again with at least one of R₁, R₂, R₃, R₄ and R₅ being methyl or methoxy.

Preferred compounds according to Formula I are those wherein:
R₂, R₃ and R₄ are each independently a C₁ to C₄ linear or branched alkoxy or a C₁ to C₈ linear or branched alkyl and R₁═R₅═H;
R₂═R₃═R₄ and are selected from a C₁ to C₄ linear or branched alkoxy or C₁ to C₈ linear or branched alkyl and R₁═R₅═H;
R₃, R₄ are each independently a C₁ to C₄ linear or branched alkoxy or a C₁ to C₈ linear or branched alkyl and R₁═R₂═R₅═H; and
R₃═R₄ and are selected from a C₁ to C₄ linear or branched alkoxy or C₁ to C₈ linear or branched alkyl and R₁═R₂═R₅═H.

Specific preferred compounds include:
Compound 1A: R₁═R₃═R₅═OMe; R₂═R₄═H
Compound 1B: R₁═R₅═H; R₂═R₄═OMe; R₃═H
Compound 1C: R₁═R₂═R₅═H; R₃═R₄═OMe
Compound 1D: R₁═R₃═R₅═H; R₂═R₄═OMe
Compound 1E: R₁═R₅═H; R₂═R₃═R₄═OMe
Compound 1F: R₁═R₄═OMe; R₂═R₃═R₅═H
Compound 1G: R₁═R₃═R₅═H; R₂═R₄═OMe
Compound 1H: R₁═R₂═R₅═H; R₃═R₄═OMe
Compound 1I: R₁═R₂═R₅═H; R₃═R₄═OMe
Compound 1J: R₁═R₂═R₄═R₅═H; R₃═OMe

It is believed that the presence of the two acetyl groups at the C-3 position of the pentanedione function of the compounds according to the present teaching is critical for proper and efficaceous chelation.

According to the present findings, these pentanedione compounds when added to various hair treatment and/or hair care compositions lessen the damage arising from the use of or typically associated with the use of those hair treatment and hair care compositions. Additionally, they may be employed in suitable carriers as a pretreatment or preventative to lessen or prevent damage from the damaging effects of the subsequent hair treatment or hair care application or as a post treatment to counteract and/or repair damage caused by the hair treatment or hair care application. In the latter, the pentanedione compounds may be added to a conditioner or other regenerative/renourishing/nourishing hair care composition.

### DETAILED DESCRIPTION

As used herein and in the appended claims the term “oxidative hair coloring composition” refers to a ready-to-use composition which can change the color of hair and which comprises an alkalizing agent, an oxidizing agent and oxidative primary dyes. A “two-component” oxidative hair coloring composition means an oxidative hair coloring composition which is obtained by mixing a tint component and an oxidizing component shortly before or at the time of application of the composition to hair. The tint component generally comprises the oxidative primary dyes and the alkalizing agent whereas the oxidizing component comprises the oxidizing agent. A “pretreatment” refers to a composition that is applied to the hair prior to treatment with an oxidative hair coloring composition or perming or relaxing composition. The term “component” means an individual compound, ingredient or mixture of compounds or ingredients which is mixed by the user with one or more other components for preparing the ready-to-use oxidative hair coloring composition to be applied to the hair. A “user” means the person preparing the hair coloring composition for application to the hair. This person may be a professional hair stylist working in a salon, an assistant who prepares the hair coloring composition for the stylist, a person who is applying the hair coloring composition to their own hair or someone doing the same or helping them in doing so.

Additionally, as used herein the term “lift” refers to the amount of removal of the natural hair pigment that the coloring composition can provide. The amount of lift provided by different hair coloring compositions can be compared using a human natural dark hair sample and measuring the change of color achieved following application of the compositions. The change in color can be measured using well known parameters such as L*a*b* values. A composition can be said to provide a higher lift than another composition when the ΔL* value measured for a given treated sample of dark hair is higher for that composition than for another composition under the same experimental conditions. The denomination Level 2 (herein used interchangeably with “demi-permanent” or “tone-on-tone”) and Level 3 (herein used interchangeably with “permanent”) are commonly used in the hair care trade to differentiate between compositions with medium lift and those with high lif.

As used herein and the appended claims the term “oxidizing agent” refers to an electron accepting compound, or combination of electron accepting compounds, suitable for use in hair coloring compositions for removing the natural color of hair (e.g., by destroying the melanin pigment) and reacting with oxidative primary dyes to provide an oxidative hair color. The most commonly used oxidizing agent in the art is hydrogen peroxide. Similarly, an “alkalizing agent” refers to compounds or a combination of compounds suitable for raising the pH to alkaline level in hair coloring compositions, in particular to a pH between 9 and 11. The most commonly used alkalizing agent in the art is ammonia. However, “non-ammonia” alkalizing agents are also contemplated and include the alkanolamines such as monoethanolamine, diethanolamine, triethanol-amine, monopropanolamine, dipropanolamine, tripropanolamine, 2-amino-2-methyl-1,3-propanediol, 2-amino-2-methyl-1-propanol (aminomethylpropanol, AMP), 2-amino-2-hydroxymethyl-1,3-propanediol and mixtures thereof.

Unless otherwise indicated, all percentages are by weight of the ready-to-use coloring composition (i.e. as applied on head after the two or more components have been mixed) or, as appropriate, the other hair treatment composition referenced and/or being employed. Similarly, all ratios are weight ratios unless specifically stated otherwise.

Inasmuch as many of the hair treatment and hair care compositions that may be used in combination with or in conjunction with the compositions of the present invention are described in many of the citations mentioned herein, especially in the background section, and/or are well known or commercially available, it is to be appreciated that they and their teachings, including any cross referenced or related patent or patent applications mentioned therein, are hereby incorporated herein by reference in their entirety unless expressly excluded or otherwise limited. To the extent that any meaning or definition of a term in this document conflicts with any meaning or definition of the same term in a document incorporated by reference, the meaning or definition assigned to that term in this document shall govern.

According to the present invention it has now been found that certain benzylidene pentanedione compounds are efficacious in providing a number of benefits in hair care applications. Specifically, it is found that the incorporation of these benzylidene pentanedione compounds into various hair care and/or hair treatment products as well as in carriers therefore, i.e., where the sole or key active ingredient is the benzylidene pentanedione compound itself or a combination of such compounds, is effective in preventing and/or reversing hair damage directly or indirection arising from other hair care ingredients, most notably hair coloring, perming, shampooing and relaxing ingredients and compositions. Additionally, these compounds, or at least certain of these compounds, are also found to improve color retention of dyed hair as well as reduce or counteract scalp irritation and itching.

Suitable benzylidene 2,4-pentanedione compounds are those according to the Formula I as follows:

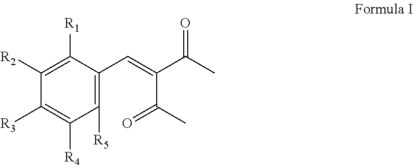

wherein R₁, R₂, R₃, R₄ and R₅ which may be the same or different, are independently H, alkyl or alkoxy, preferably H, alkyl or alkoxy, wherein the alkyl or alkoxy groups are linear or branched and have from 1 to 8 carbon atoms, preferably from 1 to 6 carbon atoms, more preferably from 1 to 4 carbon atoms, with, preferably, at least one of R₁, R₂, R₃, R₄ and R₅ being other than H. Most preferably, R₁, R₂, R₃, R₄ and R₅ are H, methyl or methoxy, again with at least one of R₁, R₂, R₃, R₄ and R₅ being methyl or methoxy.

Preferred compounds according to Formula I are those wherein:
R₂, R₃ and R₄ are each independently a C₁ to C₄ linear or branched alkoxy or a C₁ to C₈ linear or branched alkyl and R₁═R₅═H;
R₂═R₃═R₄ and are selected from a C₁ to C₄ linear or branched alkoxy or C₁ to C₈ linear or branched alkyl and R₁═R₅═H;
R₃, R₄ are each independently a C₁ to C₄ linear or branched alkoxy or a C₁ to C₈ linear or branched alkyl and R₁═R₂═R₅═H; and
R₃═R₄ and are selected from a C₁ to C₄ linear or branched alkoxy or C₁ to C₈ linear or branched alkyl and R₁═R₂═R₅═H.

Specific preferred compounds include:
Compound 1A: R₁═R₃═R₅═OMe; R₂═R₄═H
Compound 1B: R₁═R₅═H; R₂═R₄═OMe; R₃═H
Compound 1C: R₁═R₂═R₅═H; R₃═R₄═OMe
Compound 1D: R₁═R₃═R₅═H; R₂═R₄═OMe
Compound 1E: R₁═R₅═H; R₂═R₃═R₄═OMe
Compound 1F: R₁═R₄═OMe; R₂═R₃═R₅═H
Compound 1G: R₁═R₃═R₅═H; R₂═R₄═OMe
Compound 1H: R₁═R₂═R₅═H; R₃═R₄═OMe
Compound 1I: R₁═R₂═R₅═H; R₃═R₄═OMe
Compound 1J: R₁═R₂═R₄═R₅═H; R₃═OMe

The amount by which the pentanedione compound or combination of pentanedione compounds are incorporated into the hair care or hair treatment composition or, as appropriate, a carrier for direct application to the hair can vary widely and may be adjusted depending upon the nature of the composition to which it is being added and/or the extent of damage that is being addressed. For example, while many shampoos are damaging, they are certainly less damaging than a hair coloring or bleaching composition and, thus, would typically have or need less pentanedione. Similarly, as with many commercial products, there are those products that are to be characterized as being “intense” or “high strength” so as to address more highly damaged hair and, presumably, will have more of the beneficial active ingredient: in this case the pentanedione or combination of pentanediones.

Generally speaking, the amount of the pentanedione compound(s) to be incorporated into a hair care or treatment composition or a suitable carrier will be from about 0.01 to about 15, preferably from about 0.05 to about 5, more preferably from about 0.1 to 3 weight percent based on the weight of the composition or carrier to which it is added. Again, higher or lower amounts may be used depending upon the nature of the hair treatment or hair care composition whose adverse effects is to be addressed and/or the underlying damage to the hair to be treated. Again, more severely damaged hair will require or benefit from higher concentrations whereas an everyday conditioner may require a lesser level to help maintain hair quality.

As noted above, the pentanedione compounds may be incorporated into any hair care composition, so long as it does not adversely affect the efficacy and/or objective of that hair care composition hair. If it does, then the pentanedione should be incorporated into a pretreatment or post treatment composition, as appropriate. Similarly, depending upon the nature of the hair treatment or hair care products, even if combinable, it may be preferred to employ the pentanedione in a pretreatment or post treatment composition, again, as part of another hair care product, e.g., in a conditioner to be used following shampooing, or on its own as the key active. When incorporated into a conventional hair treatment or hair care composition, the user should simply follow the directions of use for that hair treatment or hair care composition as the presence of the pentanedione should not necessitate a change in the use of those products.

Inasmuch as the hair treatment and hair care compositions are well known and widely available and inasmuch as suitable carriers for hair care and hair treatment actives are likewise well known and widely available, rather than go on and on with listing the multitude of such products and their components and make-up, Applicants will instead describe a few of the key types of hair treatment and hair care compositions with which the compounds and/or compositions of the present invention may be employed. However, simply because certain types or hair care treatments and hair care compositions and/or their constituents are not specifically described herein is not to be interpreted as their not being applicable or within the scope of the present teachings and claimed invention, for they most certainly are. Similarly, their absence is not be presumed as an indication that they are less important or less relevant for that too is not the case. Again, Applicant has elected to present this abbreviated specification in light of the well-known nature of such compositions and ingredients.

That said, according to one aspect of the present invention hair treatments and/or hair care compositions which may incorporate the pentanedione compound or compounds or with which said pentanedione compound or compounds may be used include those comprising at least one non-ammonia alkalizing agent, as defined above. Exemplary and preferred non-ammonia alkalizing agents include monoethanolamine (MEA), diethanolamine, triethanolamine, monopropanolamine, dipropanolamine, tripropanolamine, 2-amino-2-methyl-1,3-propanediol, 2-amino-2-methyl-1-propanol (a.k.a. aminomethylpropanol, AMP), 2-amino-2-hydroxymethyl-1,3-propanediol and mixtures or combinations of any two or more of the foregoing. Monoethanolamine (MEA) or aminomethylpropanol (AMP) are commonly used in ammonia-free hair dye products and are among, if not the, preferred non-ammonia alkalizing agents. They may be used alone or in combination with each other or one or more other alkalizing agents, especially other non-ammonia alkalizing agents. Especially preferred is monoethanolamine, alone or in combination with one or more other non-ammonia alkalizing agents.

It is also contemplated that the compositions according to the present teachings may include or be used in combination with or in conjunction with one or more alkalizing agents which are not non-ammonia alkalizing agents, but at relatively low levels, for example less than 0.5% by weight. Most preferably the compositions according to the present teachings are substantially free of ammonia: i.e., are free of ammonia or only contain trace or negligible quantities, especially quantities that would otherwise provide an alkalizing effect.

Similarly, the compositions and methods of the present invention may be used in combination with or in conjunction with an oxidizing component, which comprises an oxidizing agent. The oxidizing component may be any usual oxidizing composition known in the art, especially for hair coloring and/or treatment. In particular, the oxidizing component may be an oil-in-water emulsion of hydrogen peroxide (H₂O₂). The oxidizing agent may be more generally selected from hydrogen peroxide, sodium periodate, urea peroxide, melamine peroxide, perborates, percarbonates, perphosphates, persilicates, persulphates, peroxidises and mixtures thereof.

In those hair coloring compositions or products that comprise two or more parts or oxidizing components and employ an oxidizing agent in each, the first oxidizing agent may be the same as or different than the second oxidizing agent and is preferably in both cases hydrogen peroxide. The concentration of the oxidizing agent may be the same or different in the first and second oxidizing component, however it may be preferred that a higher concentration in oxidizing agent is used in the second oxidizing component in order to maintain a certain concentration on-head. Usually, in a standard two-component oxidative system, non-ammonia based oxidative hair composition, the concentration of hydrogen peroxide may range from 1% to 3% by weight of the composition. For example, a commercially available hair coloring product will usually be mixed in 1:2 ratio (by weight) with a 4% or 1.9% H₂O₂ emulsion thus resulting in a 2.7% H₂O₂, and 1.3% concentration on-head.

When a hair coloring treatment includes a third component, the second oxidizing component may be the same as the first oxidizing component used in the two-component oxidative composition. The oxidizing agent may however be more concentrated in the second oxidizing component than in the first oxidizing component, especially when the relative amount of the oxidizing component in the oxidative composition comprising the third component is lower due to the dilution effect caused by the third component. Here, the concentration of H₂O₂ in the oxidative coloring composition obtained with the third component may be, for example, from 1% to 6%, in certain embodiments from 2% to 4%, in particular from 2.5 to 3.5%. Using too high a concentration of hydrogen peroxide or other oxidizing agent may not provide more lift while being damaging for the hair fiber. The second oxidizing component may comprise hydrogen peroxide as oxidizing agent in a higher concentration than, in the first oxidizing component, for example from 3 to 10% by weight of the second oxidizing component, in particular from 6 to 9%.

When the third component is used, the oxidative coloring compositions of the invention may preferably display a weight ratio of alkalizing agent to oxidizing agent (in particular a ratio of non-ammonia alkalizing agent, especially MEA: H₂O₂) which is higher than 1.1, preferably between 1.1 and 4.0, more preferably between 1.2 and 3.0, as this was found to provide a good balance between high lift and acceptable oxidative hair damages.

The compositions disclosed herein can further comprise a source of radical scavenger. As used herein the term radical scavenger refers to a species of compounds or an active site of a compound that can react with a carbonate radical to convert the carbonate radical by a series of fast reactions to a less reactive species, i.e. a carbonate radical scavenger. Radical scavengers can be selected from the classes of alkanolamines, amino sugars, amino acids, esters of amino acids and mixtures thereof. For example, the following compounds can be employed as radical scavengers: ethylamine, monoethanolamine, 2-methoxyethylamine, 3-amino-1-propanol, 4-amino-1-butanol, 5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, sarcosine, serine, glutamic acid, tryptophan, morpholine, piperidine, and mixtures thereof, and the salts such as the potassium, sodium and ammonium salts thereof and mixtures thereof.

Radical scavengers are especially desirable in hair dyeing compositions and generally are present in an amount of from about 0.1% to about 10% by weight. In another aspect of the invention, the radical scavenger is present in an amount from about 1% to about 7% by weight. When the carbonate ion is present in a hair dye composition, the radical scavenger is preferably present at an amount such that the weight ratio of radical scavenger to carbonate ion is from 2:1 to 1:4. Although not required, the radical scavenger is preferably selected such that it is not an identical species as the alkalizing agent. Further, the radical scavenger can be formed in-situ in the hair dyeing compositions prior to application to the hair fibers.

Generally speaking, water is the preferred principal solvent, carrier or diluent for the hair treatment and hair care compositions according to the present invention. As such, the compositions according to the present invention may include one or more solubility enhancers as defined above. Generally, two preferred classes of solubility enhancers are solvents and surfactant systems. Preferred solvents are miscible with water and innocuous to the skin. Solvents suitable for use herein include C₁-C₁₀ mono- or polyhydric alcohols and their ethers, glycerin, with monohydric and dihydric alcohols and their ethers, especially those whose alcoholic residues contain 2 to 6 carbon atoms. A particularly preferred group of solvents includes ethanol, isopropanol, n-propanol, butanol, propylene glycol, ethylene glycol monoethyl ether, hexylene glycol, diemethyl isosorbide, PEG-300, PEG-400, PEG-400/water (1:1) and mixtures thereof.

The solvents may be present in the composition or, if appropriate, the precursor mixture at a level of from about 0.1 to 20 wt %, preferably from about 0.1 wt to about 15 wt % and most preferably from about 0.5 wt % to about 10 wt % based on the total weight of the composition or precursor mixture.

A second class of solubility enhancers useful in the present invention is the surfactants. A particularly suitable class of surfactants is cationic surfactants. One type of preferred cationic surfactant is amine based and includes alkyl amines, alkylethoxy amines, ethoxylated alkyl amines and alkyl alkanol amines. Preferred alkyl groups have 1 to about 22 carbon atoms and can have a mixture of chain lengths, e.g., methyl and hexadecyl. The term amines include primary, secondary, tertiary and quaternary amines.

A second type of preferred cationic surfactant is amidoamines and includes C₁₂-C₂₂ alkyl or alkylethoxy mono, di and higher (poly)amidoamines which can be ethoxylated or unethoxylated. Examples include sodium dimethylaminopropyl coca-aspartamide, cocoamidopropyl dimethylamine, olivamidopropyl dimethylamine, soyamidopropyl dimethylamine, tallowamidopropyl dimethylamine, stearamidoethyl dimethylamine and mixtures thereof.

Another preferred class of surfactant that is suitable for use as a solubility enhancer is nonionic surfactants. This class includes long chain fatty alcohols, mono, di and triglyceride and their derivatives, long chain (C₁₂-C₁₈) alcohol ethoxylates and mixtures thereof. Examples include: steareth 20, oleth 10, laureth 4, PEG-400, PEG-12 glyceryl dioleate, glycerol stearate, sorbitan oleate, PPG-9 buteth-12 etc. and mixtures thereof.

The level of surfactants used as solubility enhancers in the dye precursor mixture can generally range from 0.1 wt % to about 30 wt %, preferably from about 0.2 wt % to about 20 wt % and most preferably from about 0.25 wt % to about 15 wt % based on the total weight of the composition or, as appropriate, the precursor mixture.

Both solvents and surfactants can and often are combined to achieve the desired state of solubility of the primary intermediate and coupler in a dye precursor mixture. However, it has been found that the type and level of solubility enhancer affect the ability of the oxidative dyes to absorb into the hair fibers and be retained after development. Although this can be difficult to predict, the optimum type and level of solubility enhancer can be determined empirically by treating a standard hair sample under controlled conditions with a precursor mixture and developing the color with an oxidizing agent.

Thickeners may be optionally included in the hair treatment and hair care compositions of the invention. For example, they may be incorporated into an oxidation hair colorant composition and/or hair developer composition, especially in the hair dye precursor part and the hair color developer parts of the invention, Long chain fatty alcohols having from about 11 to about 22 carbon atoms in the long fatty chain can be thickener constituents of the compositions of this invention. These alcohols can be used alone, or in admixture with each other. When included in the compositions, these alcohols are preferably present at from about 0.5 to about 10 weight percent of the composition, and more preferably at from about 2 to about 8 weight percent.

Exemplary thickeners include lauryl alcohol, oleyl alcohol, myristyl alcohol, stearyl alcohol, behenyl alcohol and the like, and mixtures thereof. In addition, mixtures of natural or synthetic fatty alcohols having fatty chain lengths of from about 11 to about 18 carbons are also useful. Several such mixtures are available commercially, and are exemplified by the material containing a mixture of synthetic alcohols with 12 to 15 carbons in the alkyl chain sold under the trademark NEODOL 25 by Shell Chemical Company, and the material containing a mixture of synthetic alcohols with chain lengths of 12 to 16 carbons sold under the trademark ALFOL 1216 Alcohol by Conoco Chemicals.

Alternatively or in addition thereto, thickening agents suitable for use in the compositions herein may also be selected from oleic acid, cetyl alcohol, oleyl alcohol, sodium chloride, cetearyl alcohol, stearyl alcohol, synthetic thickeners such as CARBOPOL, ACULYN® 28, STRUCTURE 2001, 3001, and XL, and ACROSYL® and mixtures thereof. Preferred thickeners for use herein are ACULYN® 22, steareth-20 methacrylate copolymer; ACULYN® 44 polyurethane resin and ACUSOL® 830, acrylates copolymer that are available from Rohm and Haas, Philadelphia, Pa., USA. Additional thickening agents suitable for use herein include sodium alginate or gum arabic, or cellulose derivatives, such as methylcellulose or the sodium salt of carboxymethylcellulose or acrylic polymers.

Fatty alcohols of the above discussed carbon chain lengths which are ethoxylated to contain an average of one or two moles of ethylene oxide per mole of fatty alcohol can be used in place of the fatty alcohols themselves Examples of such useful ethoxylated fatty acids include ethylene glycol cetyl ether, polyoxyethylene (2) stearyl ether, and the like; the exemplary compounds having CTFA Dictionary names of Ceteth-1 and Steareth-2, respectively.

The compositions of the present invention may also include any number of optional, thought typically conventional, additives for hair treatment and hair care compositions. Exemplary additives include: mildness enhancers such as cholesterol and its derivatives, hair swelling agents, anticaking agents, antioxidants, binders, biological additives, bulking agents, chelating agents not belonging to benzylidene pentanediones, chemical additives, colorants, cosmetic astringents, cosmetic biocides, denaturants, drug astringents, emulsifiers, film formers, fragrance components, humectants, opacifying agents, plasticizers, preservatives, propellants, reducing agents, solvents, foam boosters, hydrotropes, solubilizing agents, suspending agents (nonsurfactant), sunscreen agents, ultraviolet light absorbers, photostabilizers and viscosity increasing agents (aqueous and nonaqueous), and hair fiber lubricants. Examples of other functional classes of materials useful in the art include solubilizing agents, sequestrants, amino acids, ingredients that impart shine, hydrolyzed proteins and the like.

It may also be advantageous to include agents that condition the hair to further improve compatability and impart a silky/moisturized feel to the hair after it dries. Such agents include fatty long chain amines and their derivatives, silicones such as dimethicone and amodimethicone, long chain fatty alcohols and mixtures of these materials. One or more compounds of the present inventive compositions can also be included in the conditioners. Such conditioners can be incorporated in either the precursor mixture, the developer mixture conditioners can also be packaged separately when kits are employed.

As noted earlier, the pentanedione compounds may be used in any number of hair care and hair treatment products, each employed in their conventional methodology. Accordingly, the following discussion is presented for illustrative purposes only as hair coloring is but one of the many applications to which the present teachings may be applied. In the following discussion, the pentanedione may be incorporated into one or more of the following components or elements of the hair coloring composition and/or after treatment or applied as a separate pretreatment or after treatment.

Application of a hair coloring composition to the hair may be undertaken in several ways. For example, a hair coloring composition may be applied to the whole head of hair of an end user, i.e., that hair all over the head from the root of the hair to the tip of the hair. Alternatively, application of the hair coloring composition may take place on the root portion of the hair. The application to the root portion of the hair may still be over the entire head of the end user, but application of the hair coloring composition is applied only to the section of hair closest to the head (root portion), which is between about 0.01 mm to about 4 mm from the scalp of the head. Also, application may take place on a portion of hair. Application of a portion of hair is commonly referred to as high-lighting or low-lighting. The portion of hair may be physically separated from the whole head of hair in a hair bundle or may be a smaller portion of hair than the whole head of hair. A hair bundle may be physically separated from a whole head of hair by a device including a plastic cap through which hair bundles are formed when hair is pulled through orifices in the plastic cap, metal foils encompassing a hair bundle, strand separators applied to hair at the root portion, or similar devices.

When present, an optional conditioning agent can be provided in an additional container. In the latter case, the conditioner can be mixed immediately before use and applied together with the other components, or the content of the additional container can be applied (after an optional rinse step) as a post-treatment immediately after the hair coloring composition.

According to one method for oxidatively coloring hair, the method comprises mixing a tint component and an oxidizing component and optionally a third component comprising a second non-ammonia alkalizing agent together to form a hair coloring composition, applying the hair coloring composition to the hair to form a treated hair surface, waiting for a period of 15-60 minutes, such as 20-30 minutes, and then removing the hair coloring composition from the treated hair surface.

The methods of coloring hair also may further comprise working the hair coloring composition into the treated hair surface by hand or by a tool for a few minutes to ensure uniform application to the entire treated hair surface. The hair coloring composition remains on the treated hair surface while the end hair color develops for a time period of 15 to 60 minutes to form oxidatively colored hair. The user then rinses the oxidatively colored hair thoroughly with tap water and allows it to dry and/or styles the oxidatively colored hair.

Another embodiment of the present invention relates to a method of hair treatment, whereby the hair fiber conditioning composition is applied to the hair and worked for a few minutes (to insure uniform application to all of the hair). The composition is then allowed to remain on the hair for a time period of less than about 20 minutes, preferably less than about 15 minutes, more preferably from about 10 seconds to about 10 minutes. The user then rinses his/her hair thoroughly with water and allows it to dry and or styles the hair as usual.

In another embodiment of the present invention the oxidative hair dye or bleaching compositions may comprise as an optional fourth component a color refresher composition. Such color refresher compositions comprise at least one pre-formed dye and may contain one or more compounds of present inventive compositions and may be applied to the hair immediately after the oxidative color i.e. from about 1 minute after oxidative hair dye or bleach application to 60 days after the application. Such color refresher compositions can be used to increase the initial color obtained and or boost the color during the wash and style cycle until the next oxidative coloring or bleaching event. Alternatively the color refresher composition may be comprised within the fiber or hair fiber conditioning composition thereby ensuring a color boost with each application of the fiber or hair fiber conditioner treatment.

### EXPERIMENTAL

### Example 1

A comparative study was conducted of the Cu²⁺ chelation ability of EDTA (Ethylenediamine tetraacetic acid), EDDS (Ethylenediamine-N,N′-disuccinic acid) and 3-(3,4,5-trimethoxy benzylidene) 2,4-pentanedione (Compd. 1E) in the presence of excess Ca²⁺. The study was conducted in accordance with the methodology disclosed in “Novel Fluorometric Assay for Hydroxyl Radical Prevention Capacity Using Fluorescein as the Probe”, B Ou, M Hampsch-Woodill, J Flanagan, EK Deemer, R Prior, D Huang, J. Agric, Food Chem: 50(10); 2772-2777, 2002.

The Fenton reaction was initiated with the mixture of Cu²⁺, CaCl₂ and hydrogen peroxide at alkaline condition. Hydroxyl radicals were generated by the Fenton reagent mixture, oxidative damage was monitored by fluorescein (FL), a fluorescence probe sensitive to hydroxyl radicals. The loss of fluorescence intensity was an index of oxidation. Thus, the slower the fluorescence decay, the stronger the chelation for Cu²⁺. Fluorescence decay of fluorescein (FL) was barely noted in the presence of 200 μM each of Compound 1E (present invention), EDDS and EDTA, respectively, Other ingredients are: [CuCl₂]=230 μM, [H₂O₂]=0.055 μM, [CaCl_{2]=}2.3 mM, and [FL]=6.20×10⁻⁸ M. A second experiment with Compd 1E where equimolar amounts (230 μM) of Compd 1E and Cu²⁺ ion were present shown that the Cu²⁺ was almost completely chelated to prevent the Fenton reaction from occurring Results are summarized in the Table 1 This study clearly shows that Compd. 1E has little attraction for calcium but has excellent chelating ability for copper, leaving it unavailable for radical making: thereby reducing hair fiber and scalp damage.

TABLE 1
Cu²⁺ Chelation in the presence of
ten-fold excess amount of CaCl₂

| | % Remaining | % Remaining | % Remaining |
|---|---|---|---|
| | of FL | of FL | of FL |
| | (20 min) | (40 min) | (80 min) |
| Blank | 72.6 | 46.9 | 22.2 |
| Compd. 1E | 95.2 | 92.7 | 90.5 |
| Compd. 1E | 100.0 | 99.5 | 99.1 |
| (1:1 molar | | | |
| ratio w/CuCl₂) | | | |
| EDTA | 57.5 | 24.9 | 5.4 |
| EDDS | 78.5 | 56.4 | 30.4 |

### Example 2

A second series of experiments is conducted as described in Example 1 but replacing 3-(3,4,5-trimethoxy benzylidene) 2,4-pentanedione (Compd. 1E) with 3-(2,4,5-trimethoxybenzylidene) 2,4-pentanedione (Compd 1A), 3-(3,4-dimethoxybenzylidene) 2,4-pentanedione (Compd 1I) and 3-(4-methoxy-benzylidene) 2,4-pentanedione (Compd 1J). It is anticipated that the same results as attained with Compd 1E will be found.

### Example 3

### Hair Coloring Products

Several different kinds of hair coloring products are available. Hair coloring can be applied through coloring shampoos or hair dyes. The hair coloring products can be categorized in several types, which include the permanent, demi-permanent, semi-permanent, and temporary. The permanent coloring lasts until the hair grows out, demi-permanent lasts up to 24 washes, semi-permanent lasts for six to eight weeks, and temporary lasts for one to three washes.

A permanent hair coloring product is formulated with two different components: a precursor-coupler base and an oxidizing base. The precursor-coupler base consists of surfactants, alkaline, reducing agent, precursors, couplers, and water: an exemplary formulation for which is shown in Table 2. The oxidizing base consists of an oxidizing agent (e.g. peroxide), stabilizer for the peroxide, and surfactants: an exemplary formulation for which is shown in Table 3. Since the peroxide is unstable in alkaline solution, the precursor-coupler base and the oxidizing base have to be formulated separately for product storage. Though not shown, either or both of these components could have formulated therein a pentanedione according to the present invention.

TABLE 2
Sample precursor-coupler base ingredients

| | Weight % of ingredient for desired hair color | | | |
|---|---|---|---|---|
| Ingredient | Dark brown | Light brown | Red | Black |
| Dodecyl benzene sulfonate | 14.0 | 14.0 | 14.0 | 14.0 |
| (50%) | | | | |
| Cocodiethanolamide | 9.0 | 9.0 | 9.0 | 9.0 |
| Neodol 91-2.5 | 6.0 | 6.0 | 6.0 | 6.0 |
| Ammonium hydroxide | 6.0 | 6.0 | 6.0 | 6.0 |
| Sodium sulfite | 0.3 | 0.3 | 0.3 | 0.3 |
| p-Phenylenediamine | 0.4 | — | — | 0.4 |
| o-Aminophenol | 0.3 | 0.4 | — | 0.2 |
| p-Aminophenol | — | 0.4 | 0.4 | — |
| 4-Methyl-5-aminophenol | — | — | 0.4 | — |
| m-Aminophenol | — | — | — | 0.2 |
| Water | 64.0 | 63.9 | 63.9 | 63.9 |

TABLE 3
Sample oxidizer base ingredients

| Ingredient | Weight % of ingredient |
|---|---|
| Hydrogen peroxide (30%) | 50.0 |
| Dodecyl benzene sulfonate | 33.0 |
| (50%) | |
| Phosphoric acid | 1.0 |
| Water | 16.0 |

A demi-permanent hair coloring is similar to the permanent hair coloring in terms of oxidative dyes but resembles the lasting properties of a semi-permanent hair color. The amount of peroxide is less and therefore offers less damage to the hair.

Tables 4A and 4B present formulations for a permanent hair dye product comprising the hair dye formula and developer formula, wherein pentanediones according the present teachings are incorporated into the dye formula. Three formulations are prepared in which the pentanedione is Compd 1A, Compd 1C and Compd 1E presented as a composition wherein the pentanedione is solubilized in PEG-300 or PEG-400 or PEG-400/water (1:1) etc.

TABLE 4A
Permanent Hair Dye Formula

| | |
|---|---|
| INCI Name | % w/w |

Phase A-1

| | |
|---|---|
| Water (demineralized) | QS |
| pentanedione | 0.1 to 0.3% |
| Glycerin | 3.00 |
| Sodium Sulfite | 0.20 |
| Resorcinol | 0.50 |
| Ethanolamine | 0.50 |
| Linoleamidopropyl PG- Dimonium | 0.50 |
| Chloride Phosphate | |

Phase A-2

| | |
|---|---|
| Carbomer | 0.40 |
| Poiyquaternim -39 | 0.20 |

Phase B

| | |
|---|---|
| Ammonium Hydroxide (50%) | 5.00 |

Phase C

| | |
|---|---|
| Glyceryl Stearate | 2.50 |
| Cetearyl Alcohol | 3.00 |
| Sodium Laureth Sulfate | 2.00 |
| Octyldodecanol | 2.00 |
| Ceteareth-20 | 1.00 |
| Oleic Acid | 1.00 |
| Sodium Cetearyl Sulfate | 1.00 |

Phase D

| | |
|---|---|
| m-Aminophenol | 0.75 |
| Sodium Sulfate | 0.25 |
| Toluene 2,5-Diamine Sulfate | 0.75 |
| 2-Amino-4-Hydroxyethylaminoanisole | 0.10 |
| Sulfate | |

Phase F

| | |
|---|---|
| Citric Acid | QS |
| Benzoic Acid | 0.20 |
| Total | 100.00 |

TABLE 4B
Cream Developer Formula

| | |
|---|---|
| INCI name | % w/w |

Phase A-1

| | |
|---|---|
| Water (demineralized) | QS |
| Propylene Glycol | 2.00 |
| Disodium Pyrophosphate | 0.20 |
| Etidronic Acid | 0.50 |
| 2,6-Dcarboxypyridine | 0.25 |

Phase A-2

| | |
|---|---|
| Hydrogen Peroxide | 5.00 |

Phase B

| | |
|---|---|
| Potassium Hydroxide (50%) | 2.00 |

Phase C

| | |
|---|---|
| Cetearyl Alcohol | 4.00 |
| Mineral Oil | 4.00 |
| Ceteareth-20 | 2.00 |

Phase D

| | |
|---|---|
| Citric Acid | QS |
| Potassium Benzoate | 0.20 |
| Total | 100.00 |

### Example 4

A post treatment for color treated hair is prepared which serves as both a hair and scalp conditioner, relieving hair damage caused by the color treatment as well as the itching associated with the irritation of the scalp from the hair coloring process and treatment. The formula is presented in Table 5.

This product is formed according to the following procedure: Add Phase B to A. After fully hydrating the combination of A and B, heat the combination to 75° C. Separately combine the ingredients for C and heat to 80° C. The add C to the combination of A and B with mixing. Under continuous propeller agitation, cool the mixture to 40° C. Finally, add D to the mix and mix well under agitation until a uniform.

TABLE 5
Hair and Scalp conditioner for color treated hair

| | |
|---|---|
| INCI name | % w/w |

Phase A

| | |
|---|---|
| Water (demineralized) | 82.00 |

Phase B

| | |
|---|---|
| Hydroxyethylcellulose | 1.00 |
| Glycerin | 2.00 |
| Cetrimonium Chloride | 0.50 |
| Phenoxyethanol | 1.00 |
| Potassium Sorbate | 0.25 |

Phase C

| | |
|---|---|
| Glycery Stearate | 1.00 |
| Cetary Alcohol | 5.00 |
| Cetyl Esters | 0.50 |
| Behentrimonium Chloride | 1.00 |
| Isosorbide Dicaprylate | 2.00 |
| Caprylic/Capric Triglyceride | 1.50 |
| Trimethoxybenzylidene Pentanedione | 0.50 |
| (Compd. 1E) present invention | |

Phase D

| | |
|---|---|
| Panthenyl Hydroxypropyl | 0.30 |
| Steardimonium Chloride | |
| Panthenol | 0.30 |
| Hydrolyzed Rice Protein | 1.00 |
| Sodium Hydroxide | 0.15 |

### Example 5

### Dry & Wet Hair Combing

In order to demonstrate the effect of the pentanedione as a preventative/reparative ingredient for hair coloring products, a series of tests were performed in which hair tresses weighing approximately 3 g and measuring 8″ in length and 1″ in width were procured from International Hair Importers & Products (Glendale, N.Y.) were bleached using a standard protocol. One half of the hair samples were then dyed using a commercial hair coloring product (Revlon ColorSilk (ammonia-free) #43 Medium Golden Brown for Colorant and Developer) with half of those tresses being colored with the hair coloring product as is (Colored) and the other half being colored with the hair coloring product to which 0.5% by wt. of Comp 1E (as a 20% solution inPEG-400) was added to the colorant (the “Modified Colored”). Coloring was performed in accordance with the manufacturer's directions. After dyeing, the hair tresses were allowed to stand for 30 min before any further processing. Thereafter one half (8) of the Colored and one half (8) of the Modified Colored tresses were dried for the same period of time under the same drying conditions.

All of the hair tresses, wet and dry, were then subjected to combability testing to assess the combing force needed to comb through the hair. Combing forces were determined using an Instron tensile tester to measure frictional forces while a hair tress is pulled through a comb in accordance with the widely-used method of Garcia & Diaz (JSCC, 27, (1976) 379-398 Combability Measurements on Hair). Six combing strokes were performed per tress with eight replicate hair tresses for each test to ensure statistical relevance. JMP™ analytical software was used to perform the statistical analysis. Statistics were performed using the students t-test at the 95% confidence level.

The results of the wet combing test are shown in Table 6 and the results of the dry combing test are shown in Table 7. In the latter, only sever replications were used in setting forth the final results as one tress in each provided a clearly anomalous result, warranting its exclusion.

TABLE 6
Wet Combing

| | | | | Standard | Standard |
|---|---|---|---|---|---|
| Treatment | | Number | Mean (gf) | Deviation | Error Mean |
| Colored | Baseline -1 | 8 | 107.21 | 19.99 | 7.07 |
| | Control | 8 | 107.54 | 20.87 | 7.38 |
| Modified | Baseline-2 | 8 | 97.32 | 16.26 | 5.75 |
| Colored | Invention | 8 | 77.60* | 11.13 | 3.93 |

*Statistically significant (p = 0.05)

As shown in Table 6, hair tresses that were colored with the modified coloring product (Modified Colored) showed a significant decrease in wet combing forces from its baseline, while the hair tresses that were colored with the unmodified coloring product (Colored) showed no difference in wet combing force from its baseline. More importantly, there is a marked difference in the wet combing forces as between the Colored and Modified Colored hair tresses.

TABLE 7
Dry Combing

| | | | | Standard | Standard |
|---|---|---|---|---|---|
| Treatment | | Number | Mean | Deviation | Error Mean |
| Colored | Baseline-1 | 7 | 138.9 | 57.52 | 20.34 |
| | Control-1 | 7 | 67.2*+ | 10.47 | 3.70 |
| Modified | Baseline-2 | 7 | 148.1 | 39.12 | 13.86 |
| Colored | Invention | 7 | 51.5*+ | 34.87 | 12.33 |

*+Statistically significant (p = 0.05)

As seen from Table 7, there is a significant decrease in dry combing forces for both the Colored and Modified Colored hair tresses over their respective baselines. Also, there is a significant difference between combing forces for the Modified Colored and Colored hair tresses as well.

Based on the results presented in Tables 6 and 7, it is clear that the pentanedione compound provided a lubriciousness and/or silkiness to the hair and/or reduced tangles in the hair as evidenced by the much lower, markedly lower in the wet comb test, combing forces. These results are consistent with the manifestation of less damage to and friction on the hair, thereby facilitating manageability and providing detangling benefits.

Similar experiments are contemplated with Compd 1I and Compd 1J with the expectation of similar results.

### Example 6

A further experiment was conducted to assess the impact of the use of hair care products containing the select pentanediones on hair which has been colored, particularly with respect to color fading. Such issues are well known and reported on, e.g., B Locke and J Jachowicz, Fading of artificial hair color and its prevention by photofilters, J Cosmet Sci, 56 407-425 (2005).

In this experiment, bleached European brown hair tresses were dyed with a commercially available medium auburn color for 30 min following the instructions provided in color kits. For testing of UVB damage, virgin dark brown hair was used. A number of the tresses were treated with a standard shampoo and conditioner whereas a second number were treated with the same except that Compd 1E was added to each. A subset of the treated tresses were then irradiated for 3 hr on each side, totaling 6 hr-equivalent to one day of UV exposure with UV radiation (200-400 nm) at 50 W/m1 using a quartz inner filter and type “s” borosilicate outer filter. At the start of the study and at intervals equivalent to every five days of UV exposure (which is after every 30 hours), tresses were treated again with the Compd 1E modified and the unmodified shampoos and conditioners. Irradiated tresses were exposed to the total equivalent of 15 days of UV exposure. Test conditions were maintained at 40° C. and 65% relative humidity. Different types of tresses were compared, including those that were dyed and not exposed to treatment or UV exposure; dyed and treated with the control shampoos and conditioners and exposed to UV; and dyed and treated with shampoos and conditioners containing the Compd 1E and exposed to UV. Comparative color loss was determined both objectively and subjectively. The total color loss (ΔE) was calculated by assessing the changes in L*, a*, b* readings. The changes in hair color indexes were calculated using the respective values after the shampoo and conditioner treatments and before UV exposure; and after the equivalent of 15 days of UV exposure. The results of the instrumental evaluations were confirmed via subjective testing.

Based on the objective testing, the total color change of the medium brown-colored hair treated with the modified and unmodified shampoos and conditioners after 15 days of UV exposure were as follows;
ΔE control-10.46
ΔE Compd 1E-7.28

The swatches were photographed and compared to dyed but untreated swatches, which represent the initial dye color, to allow for visual comparisons. Panelists were also asked to rate the color protection effects in simple comparisons of the control and the Compd 1E swatches to the untreated control. The panel evaluation demonstrated a strong correlation between the visual results and the instrumental results. On medium brown hair, 95.1% of panelists chose the tresses treated with the Compd 1E shampoo as having retained the most color. Accordingly, these results show that the pentanediones also prevent/reduce hair color degradation of colored hair, especially that which is induced by sun-induced photodegradation.

### Example 7

### Ethnic Hair Relaxer & Shampoo

To further demonstrate the breadth of the benefits attainable with the practice of the present invention, hair samples were treated with a commercial hair relaxer product (Dark and Lovely Beautiful Beginnings No-Mistake Smooth Relaxer for All Hair Types) This product has four components: the Hair and Scalp Protective Gel comprising Paraffinum Liquidum (Mineral Oil), Diethylhexyl Maleate, Isoprene Copolymer, Hydrogenated Styrene, and Butadiene Copolymer; the No-Mistake Relaxer Cream comprising water, Petrolatum, Paraffinum Liquidum (Mineral Oil), Cetearyl Alcohol, Guanidine Carbonate, PEG-75 Lanolin, Behentrimonium Methosulfate, Polyquaternium-6, Isopropyl Myristate, Isopropyl Palmitate, Cl 15510 (Orange 4), Denatonium Benzoate, Aloe Barbadensis Leaf Extract, Aloe Barbadensis Extract; the No-Mistake Cream Activator Propylene Glycol, Calcium Hydroxide, Silica, Cl 77891 (Titanium Dioxide); the Color Signal Neutralizing Shampoo comprising water, Sodium Laureth Sulfate, Polysorbate 21, PPG-5-Ceteth-10 Phosphate, Cocamidopropyl Betaine, Salicylic Acid, Polysorbate 20, Polyquaternium-7, Sodium Chloride, Hexylene Glycol, Sodium Hydroxide, Hydroxypropyltrimonium Honey, Phenolsulfonephthalein, Citric Acid; and the Deep Conditioner comprising water Propylene Glycol, Cetearyl Alcohol, Polyquaternium-37, Propylene Glycol Dicaprylate, Dicaprate, Behentrimonium Methosulfate, Sodium Benzoate, PPG-1 Trideceth-6, Stearyl Dimethicone, Chlorhexidine Dihydrochloride, 2-Oleamido-1, 3-Octadecanediol.

The commercial product was used as instructed except that a number of the hair tresses were treated with the commercial product was modified by adding Compd 1E to the relaxer and/or Compd 1E to the shampoo. In both instances, the amount of Compd 1E added was 0.5 g and was added as a 20% solution of Compd 1E in PEG400

As with the previous example, hair tresses were evaluated by a panel of six individuals and rated based upon coarseness, smoothness, etc. as manifested by easy of combing. Ratings were based upon a sliding scale wherein “+” represented the baseline before treatment of the hair, “++” represented very minor damage with minimal tangles, “+++” represented moderately damaged hair with moderate tangling, and “++++” represented severely damaged hair with significant tangling. The results are presented in Table 8.

TABLE 8
Relaxer Evaluation

| | | Control | Modified 1 | Modified 2 |
|---|---|---|---|---|
| | Baseline | Commercial | Compd 1E | Compd 1E |
| | Before | product used | added | added |
| Resultse | treatment | as-is | to relaxer | to shampoo |
| Evaluation | + | ++++ | ++ | ++ |
| by panelists | | | | |
| (6) | | | | |

As noted in Table 8, the panel evaluation concluded that the addition of the pentanedione Compd 1E to the relaxer and shampoo markedly reduced the damage caused by the use of the same product without Compd 1E. In this study, more than 80% of the panelists characterized the hair treated with the Compd 1E shampoo or relaxer as being very smooth in appearance and silky to the touch.

Without further elaboration, it is believed that one skilled in the art can, using the preceding description, can utilize the present invention to its fullest extent. Furthermore, while the present invention has been described with respect to aforementioned specific embodiments and examples, it should be appreciated that other embodiments utilizing the concept of the present invention are possible, and within the skill of one in the art, without departing from the scope of the invention. The preceding preferred specific embodiments are, therefore, to be construed as merely illustrative, and not limitative of the remainder of the disclosure in any way whatsoever.

I claim:

1. A hair care or treatment composition comprising an active hair care or hair treatment agent known to result in damage to hair or a loss in combability and at least one benzylidene 2,4-pentanedione compound having the Formula I 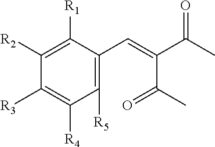
wherein R₁, R₂, R₃, R₄ and R₅ which may be the same or different, are independently H or a linear or branched alkyl or alkoxy group having from 1 to 8 carbon atoms, and wherein the benzylidene 2,4-pentanedione is present in an amount sufficient to prevent or lessen the adverse effect of the hair care or hair treatment agent.

2. The composition of claim 1 wherein at least one of R₁, R₂, R₃, R₄ and R₅ is not H and is independently selected from an alkyl or alkoxy group, which is linear or branched, and has from 1 to 6 carbon atoms.

3. The composition of claim 1 wherein at least one of R₁, R₂, R₃, R₄ and R₅ is not H and is independently selected from an alkyl or alkoxy group, which is linear or branched, and has from 1 to 4 carbon atoms.

4. The composition of claim 1 wherein R₂, R₃ and R₄ are each independently a C₁ to C₄ linear or branched alkoxy or a C₁ to Cₐ linear or branched alkyl and R₁═R₅═H.

5. The composition of claim 1 wherein R₂═R₃═R₄ and are selected from a C₁ to C₄ linear or branched alkoxy or C₁ to C₃ linear or branched alkyl and R₁═R₅═H.

6. The composition of claim 1 wherein R₃, R₄ are each independently a C₁ to C₄ linear or branched alkoxy or a C₁ to C₆ linear or branched alkyl and R₁═R₂═R₅═H.

7. The composition of claim 1 wherein R₃═R₄ and are selected from a C₁ to C₄ linear or branched alkoxy or C₁ to C₃ linear or branched alkyl and R₁═R₂═R₅═H.

8. The composition of claim 1 wherein the benzylidene 2,4-pentanedione is 3-(3,4,5-trimethoxy benzylidene) 2,4-pentanedione.

9. The composition of claim 1 wherein the hair care or hair treatment composition is a shampoo, perming composition, dyeing composition, oxidizing composition, relaxing composition, or conditioner.

10. A composition suitable for treating hair comprising:
a. an oxidizing agent and
b. a benzylidene 2,4-pentanedione compound or compounds capable of selectively chelating Cu²⁺ in presence of excess Ca²⁺ having the Formula I 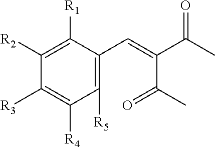
wherein R₁, R₂, R₃, R₄ and R₅ which may be the same or different, are independently H or a linear or branched alkyl or alkoxy group having from 1 to 8 carbon atoms, and wherein the benzylidene 2,4-pentanedione is present in an amount sufficient to prevent or lessen the damage caused by the oxidizing agent.

11. The composition of claim 10 wherein the oxidizing agent part of a hair coloring composition or product.

12. The composition of claim 11 wherein at least one of R₁, R₂, R₃, R₄ and R₅ is not H and is independently selected from an alkyl or alkoxy group, which is linear or branched, and has from 1 to 6 carbon atoms.

13. The composition of claim 11 wherein at least one of R₁, R₂, R₃, R₄ and R₅ is not H and is independently selected from an alkyl or alkoxy group, which is linear or branched, and has from 1 to 4 carbon atoms.

14. The composition of claim 11 wherein R₂, R₃ and R₄ are each independently a C₁ to C₄ linear or branched alkoxy or a C₁ to C₈ linear or branched alkyl and R₁═R₅═H.

15. The composition of claim 11 wherein R₂═R₃═R₄ and are selected from a C₁ to C₄ linear or branched alkoxy or C₁ to C₈ linear or branched alkyl and R₁═R₅═H.

16. The composition of claim 11 wherein R₃, R₄ are each independently a C₁ to C₄ linear or branched alkoxy or a C₁ to C₈ linear or branched alkyl and R₁═R₂═R₅═H.

17. The composition of claim 11 wherein R₃═R₄ and are selected from a C₁ to C₄ linear or branched alkoxy or C₁ to C₈ linear or branched alkyl and R₁═R₂═R₅═H.

18. The composition of claim 11 wherein the benzylidene 2,4-pentanedione is 3-(3,4,5-trimethoxy benzylidene) 2,4-pentanedione.

19. The composition of claim 11 wherein the hair care or hair treatment composition is a shampoo, perming composition, dyeing composition, oxidizing composition, relaxing composition, or conditioner.

20. A method of reducing the adverse effects of a hair care or hair treatment composition said method comprising the addition of an effective amount of at least one benzylidene 2,4-pentanedione compound having the Formula I 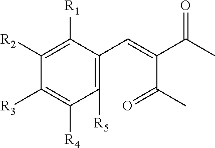
wherein R₁, R₂, R₃, R₄ and R₅ which may be the same or different, are independently H or a linear or branched alkyl or alkoxy group having from 1 to 8 carbon atoms to counteract the effects of the hair care or hair treatment composition that would manifest in the absence of the benzylidene 2,4-pentanedione.
